(19) United States
(12) Patent Application Publication
(10) Pub. No.: US 2016/0066837 A1
(43) Pub. Date: Mar. 10, 2016

(54) Personal Device and Method for Integration of Mind/Body Focus Energy

(71) Applicant: Sandra Patricia Melo, Palm Springs, FL (US)

(72) Inventor: Sandra Patricia Melo, Palm Springs, FL (US)

(21) Appl. No.: 14/891,640

(22) PCT Filed: May 6, 2014
(86) PCT No.: PCT/US14/37032

§ 371 Date: Nov. 16, 2015

Related U.S. Application Data
(60) Provisional application No. 61/825,002, filed on May 18, 2013.

(51) Int. Cl. A61B 5/00; G09B 5/00; G09B 19/00; A61B 5/01

(52) U.S. Cl. CPC A61B 5/486; A61B 5/01; A61B 5/6824; A61B 5/6829; A61B 5/6823; A61B 5/6831; A61B 5/7455; G09B 5/00; G09B 19/00

(57) ABSTRACT

The present invention provides a device and related method that uses a physical stimulus (27) to alert the user of a possible disturbance through biofeedback in response to external body temperature fluctuations outside the normal range (26) to make the user consciously aware (30) of the disturbance so the user can refocus energy and attention. The present device comprises an electric circuit (8), energy source (3), timing element, physical stimulus generator (4), programmable component which sends a physical stimulus to said user at a default or programmed time interval, and at least one thermal sensor (9) disposed to come into direct contact with the user's skin to detect abnormal changes in skin temperature and send a physical stimulus to the user.

### CROSS-REFERENCE TO RELATED APPLICATIONS

This application claims priority of U.S. Provisional Application Ser. No. 61/825,002, filed on May 18, 2013, titled ‘Personal Device and Method for Integration of Mind/Body Focus Energy,’ which application is incorporated in its entirety by reference in this application.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention is in the technical field of personal devices and associated methods for focusing energy and attention. The method and related device works by causing an interruption in the usual stream of mental activity by providing an external sensory stimulus, giving the mind something to focus on for a short moment creating a gap. More specifically, the present invention provides a device and related method that uses a physical stimulus or vibration to (1) alert the user of a possible potential disturbance and (2) to provide the user with a clear mental space to reboot or reset the mind's mental activity that will allow for a new sense of awareness in the present moment to come forth prompting a re-evaluation of the users current stance on his or her environment. The device and method can be used as a therapeutic aide with everyday applications that will work for the normal individual who just needs to focus, as well as the one in distress who is in need of a more direct, intimate, moment to moment approach to stress and anxiety.

2. Description of Prior Art

Studies have shown that when people relax or experience positive emotional states, the hands and feet of those individuals get warmer, since the blood vessels relax and allow more warmth by increased blood flow from the core of the body to the extremities. In contrast, the opposite occurs in the case of negative mental states, blood vessels constrict therefore reducing blood flow, such that blood flow would move away from the extremities. Blood flow is reduced due to the constriction of the vessels triggered by stress, fear or anxiety due to the natural built-in fight or flight response.

The physical body is greatly affected by negative emotional states, including that muscles may become weak, normal immune system functioning will be disrupted making individuals more prone to diseases, and the digestive and endocrine systems will be disrupted by the discharge of intense, low frequency, negative emotional energy into the body. In short, negative emotional states severely affect quality of lives, playing a determining factor in an individual's health, experiences, performance and overall well-being.

A process of conscious integration of mind and body is necessary for the effective interpretation, discernment and processing of the feedback or information received by the senses to maintain the flow of energy constant and interrupted.

In everyday experience, the point of convergence of the harmonious cycle in which an individual's vital energy is originated and renewed is the point in which his or her mind and body align. This alignment creates the perfect correspondence where energy or feedback received by his or her body from the external and internal environment meet to be filtered, processed and translated by his or her conscious mind becoming available to him or her to direct or focus.

The prior art patents and published patent applications provide various wearable reminder devices, energy bands and attention focusing devices using reward systems, however, none of the prior art devices or methods alone or combined teach of the device or method of the present invention. Nothing in the prior art teaches of using a device including at least one thermal sensor to detect different emotional states within the wearer, and then to send sensory stimuli in response to potential emotional biofeedback detected. Some wearable medical devices are available in the prior art, such as a fertility monitoring device made by DuoFertility™ using a basal cell temperature sensor on skin which attaches a coin-sized sensor unit to the user's body with a biocompatible adhesive patch or tape and uses a handheld reader unit to analyze the data and allows the user to transfer that data to medical professionals for further analysis. Although this prior art sensor uses a thermal skin sensor to detect biofeedback, the structure of the device, mechanism, functions and purpose are entirely different. Further, the present invention does not use a handheld reader unit to analyze data nor does it transfer that data to medical professionals for further analysis.

Unlike the prior art, the present invention does not contain a display screen for alerts, nor does it create a reward system, nor does it work through remote control. The function, purpose, use and structure of the device and method of the present invention provide a novel means to increase user awareness and focus.

The prior art provides no viable attention focusing devices for individuals with hectic schedules, those working in demanding or stressful environments, or for individuals struggling to control an addiction or a compulsive behavior looking for an effective approach to anxiety.

Further, the prior art teaches of energy bands that assist athletes, fitness enthusiasts or individuals seeking balance in their lives, but such energy bands do not work through the mechanisms, means or functions of the present device and method.

In light of the foregoing prior art devices and methods, the device of the present invention substantially diverges in design elements and the method provides distinct steps from the prior art. There is a current need for a personal device and method that is activated by sensory stimuli in combination with programmed inputs in the field of attention focusing devices and methods, which the present invention adequately fulfills.

### SUMMARY OF THE INVENTION

The present invention provides a personal device and method of focusing energy and attention. The device comprises a module or cell including two different physical stimuli components. In the preferred embodiment of the present invention the physical stimuli will be in the form of a silent vibration. One of the physical stimulus components is a temperature triggered mechanism with a built-in thermal sensor that automatically sends the sensory stimuli upon activation to provide biofeedback registered by detecting skin temperature fluctuations. The device then sends a signal to the user, for example in the form of a vibration, to alert the user that a sudden significant temperature fluctuation in the skin (outside the normal range) has been registered indicating a disturbance in the normal body biology, possibly due to the unconscious experiencing of a heighten emotion. The second physical stimulus component is a pre-programmed mechanism that sends the stimuli based on a default setting; that provides the user the option to use controllers to adjust settings to fit personal needs. For example, the default setting could provide that the vibration be sent every hour, sustained and uninterrupted for 30 seconds, with the purpose of drawing the user's mind's attention to the body long enough to engage the mind in sensory perception. The controllers allow the user to separately change the frequency, the intensity and the duration of the vibration. The two physical stimuli components are calibrated to work in sync with one another, providing the user with an integrated system of temperature biofeedback tracking and sensory perception monitoring necessary for the activation of conscious processing of information of the cycle of energy origination, renewal and conservation to be completed. However, because each of the components provides different benefits on their own, the user has the option to have one or the other operating separately on the device at any given time.

In the preferred embodiment, the device is designed to be discreetly worn on the wrist, the upper arm, the ankle or the chest area. The device can be adapted to be attached to a chain, a bracelet or a watch-like band, or can be worn as a clip attachable to clothes, underwear and jewelry. The components of the device are comprised within one cohesive mechanism, with the controllers accessible to the user of the device. Further, since the device's objective and method call for a complete intimate interaction with the user where there is no use of screens or visual stimulation, in the preferred embodiment the device is designed to be completely silent, in the form of a thin, round shape that can be easily hidden from view if desired, or that can be concealed behind a decorative face to be worn as an accessory using different attachment methods.

Although each of the two physical stimuli components within the device can be used independently to provide separate benefits, the overall purpose of the device is effectively carried out through the method of integrating the two components in two steps. First, when the device is operating in the programmed mode, the purpose is to cause an interruption in the usual stream of mental activity, giving the user's mind something to turn the focus of its attention to for a short period of time (such as the duration of time the sustained physical stimulus or vibration occurs for). Second, the built-in skin thermal sensor for temperature biofeedback provides a tool to easily monitor moment to moment skin temperature, detecting any significant fluctuations outside the normal range when conditions are stable, and then alerting the user through sending a signal through the device. This signal is to alert the user of any disturbance in the physiological processes taking place in the body that are triggered by the nervous system (fight or flight response) in response to a heightened emotional state and manifested in the body as physiological cues, such sudden skin temperature fluctuations. The combination use of the programmed activation of stimuli and the biofeedback activation of stimuli comprise the preferred operation of the present invention.

First, in the programmed mode, the wearer user can allow this programmed stimulus to remind the user to refocus energy or attention, or to open a mental space available for the insertion of a new idea, a new thought, or simply a space to reboot or reset the mind's mental activity. This could result in a refreshed, renewed, re-energized and expanded sense of awareness of the user's body, the present moment and the user's surroundings, creating a condition optimal for the deliberate re-direction of focused energy, prompting the user to re-evaluate his or her current position/stance on his or her environment, as well as his or her current physical and mental states. Further, the user can use the sustained physical stimuli to re-engage by consciously directing the focus of awareness or to dis-engage accordingly to that evaluation, keeping the user from wasting the vital energy invested in highly charged negative emotional states, thoughts and feelings. The sustained stimulus sent to the body forces the mind to direct its attention to the part of the body that is receiving the stimulus and to stay with it in the present moment for as long as the stimulus is received awakening the brain-body senses which anchors the user in the experience of mind and body in the moment. It is at this point that the user has an opportunity to identify the emotional state he/she is in (in his/her mind) and how the brain is translating that state in the body (through the senses) as specific physical sensations and feelings. This is the point of convergence of mind and body. This mind and body convergence allows the user time to engage his or her full sensory perception and to bring perception to the conscious awareness to be processed, anchoring the experience in the moment in which an individual can consciously engage his or her mind, while being aware of his or her body and discerning what is happening at the present moment, such as feelings, thoughts, and situations.

This step of providing a stimulus at a programmed interval, allows the user to reflect and consciously focus on the feedback from the outside world received by the body through the senses, rather than passively allowing this information to go directly into the brain unfiltered and unprocessed by the conscious mind. Without integrating the mind in a conscious deliberate process of interpretation and discernment of the information or feedback received as stimuli by the senses, this unprocessed information automatically goes to the brain where it is translated and sent back to the body in the form of thoughts, feelings, images, emotions correspondent to this incomplete and often distorted version of reality. Over time this information becomes automatic in the brain shaping the body's conditioned responses to the next experience and the next becoming a template. Moment after moment, this is the process by which individuals create their habits and experiences, through the way they feel, think and act or react in their lives. Whether an individual is an executive, an athlete, or a regular person, that individual's environment calls to react to it. A process of integrating mind and body is necessary for a successful conscious processing of information, enhancing the ability to effectively select the desirable elements from the environment and highlight them by directing the energy of focused awareness to them. This process of selective filtering may allow for an intentional direction of focused energy and heightened awareness resulting in optimal physical and mental correspondence, in which the user is able to exert the least effort to get the greatest results.

Energy is typically expended when concentration is required in any situation. The use of the present device aids the user in the engagement of the natural process of mind and body connection to renew and conserve energy by helping the user to adjust the level of intensity of focus energy investment based on the specific needs and situation; as energy is being expended when the brain is focusing on an activity. A mind that is not aware of the energy of the body is not discerning information and therefore is focusing on stimuli, in a constantly reactive state, causing the brain to be overactive, to be restless and the body to be in constant alert mode. If the mind is aware of the body's energy, it is not focused and its attention is not on stimuli, but rather the mind is observing and registering but is not engaged (energy is not being consumed), it is relaxed and so is the brain. Being aware of the body's energy focus, relaxes the mind and the brain resulting in an overall sense of wellbeing manifested in the body in more concentration, alertness, better judgment, improved problem solving skills, strengthening of the immune system, calmness and a sense of connection and inner joy. In contrast, intense focused activity without the grounding of awareness leaves the body depleted of its vital energy throwing the body out its natural balanced state into a state in which its energy is spent at a faster rate than it is renewed causing a disruption in the normal flow of energy. This results in all kind of maladies both mental and physical.

The second physical stimuli component of the present device, the built-in skin thermal sensor for temperature biofeedback, provides a tool to easily monitor moment to moment skin temperature, signaling any significant fluctuations outside the normal range when conditions are stable, alerting the user of a possible disturbance in the physiological processes taking place in the body that are triggered by the nervous system in response to a heightened emotional state and manifested in the body as physiological cues such sudden skin temperature fluctuations, among other things. The thermal sensor automatically triggers the alert (i.e. vibration) when there is a skin temperature fluctuation outside normal ranges as a potential indicator of an intense emotional state. Significant temperature fluctuations outside the normal range herein refer to body temperature fluctuations bringing the body temperature below 91.4 degrees Fahrenheit, which may be indicative of a potential heightened negative emotion. The sensor takes into account that normal body temperature fluctuations throughout the day range from 2 to 4 degrees Fahrenheit for various reasons, including after a meal is consumed.

In the preferred embodiment of the present invention, the thermal sensor is a thermistor, which unlike regular thermometers that use a scale of mercury, a thermistor uses a highly thermally sensitive resistor, which will exhibit a precise change in electrical resistance when subjected to a corresponding change in body temperature. The ideal body temperature is regulated at 37 degrees Centigrade or 98.6 Degrees Fahrenheit, where the average normal body temperature associated with a relaxed state will be within the range of 93.2 to 98.6 degrees Fahrenheit. An external body temperature of 91.4 to 93.2 degrees Fahrenheit indicates a range between very relaxed and slightly stressed. The device and method disclosed herein will register and respond to external body temperatures in the range of 91.4 to 86 degrees Fahrenheit, as these external body temperatures are indicative of the presence of a stressed state for a healthy individual under stable conditions. A healthy individual under stable conditions is defined herein to refer to an individual of good health, not taking medications that alter the body temperature, in the standard environment of 77 degrees Fahrenheit, and that the individual is not engaged in physical activity that will affect body temperature.

The present device and method provide a novel approach to biofeedback that is completely intimate and personal, such that when the thermal sensor is activated, the device alerts the user, providing the user with the option to stop, observe, and consciously identify and assess his or her feelings and experiences. Biofeedback may be used to improve health or performance and the physiological changes often occur in conjunction with changes to thoughts, emotions and behavior.

The use of the built-in skin thermal sensor feature of the present device aids the user to regain awareness of, identify and get acquainted over time with the physiological cues signaling a possible unconscious negative internal state causing havoc in the user's system, affecting health, weakening the body and draining its energy. By becoming aware of these cues, users can track negative emotional states they experience unconsciously and react to automatically; breaking the cycle by becoming consciously aware of negative states and their physical manifestations and starting to change them. Through use of the device and method, the user engages full awareness of positive and negative feelings at the time he or she receives the sensory signal based on a change in body temperature. Full awareness of feelings is crucial to effectively identify the often “subtle” signs of negative emotional states, as emotional states filter and distort perception. Automatic responses to negative emotional states take up great amounts of vital energy, weakening and altering the whole body system's normal functioning, effecting mental and physical health, ability to focus, think clearly and perform (both physically and mentally) to the best of one's abilities.

By activating awareness through biofeedback and interval activated generation of stimuli, the present invention provides the platform, tools and methods to aid the user to start the process to regain full awareness of his or her environment, body, emotions and feelings that constitute to the blueprint of his or her experience. Further, the present device and method allow the user to ground that experience in the present moment, and with this awareness to increase his or her ability to consciously draw and direct or focus vital energy towards his or her personal interests, goals and overall well-being.

The present method and device are intended for use in the areas of stress management, developing problem solving skills, overall performance enhancement, assisting in addiction control, controlling a compulsive behaviors, dealing with anxiety, increased performance for athletes and fitness enthusiasts and for individuals seeking balance, more clarity of mind, and those seeking to experience an expanded sense of awareness and well-being.

Individuals with hectic schedules, in demanding and stressful environments, and/or under intense mental performance expectations, can benefit from use of the present invention. Whether they are looking for an integral effective alternative to appropriately address stress management, problem solving skills or overall performance enhancement, the device and method allow users to position themselves in mind/body alignment, where their energies balance because they are in a state of equilibrium, resulting in relaxation of the mind, being free from the gravitational pull of these energies, creating clarity, as well as the ability to think and solve problems efficiently.

Individuals struggling to control an addiction, a compulsive behavior, or those looking for an effective approach to anxiety, benefit from the device mechanism and method provided herein, as a way to monitor and track the presence of the potential mental disturbances leading to the automatic engagement in the compulsive or destructive behavior, providing users with an opportunity to disengage by becoming aware, breaking the cycle, drawing the mind out the compulsive thought patterns that trigger the behavior, weakening them and allowing the process of new patterns to begin to take root and to strengthen over time freeing users from the grip of the compulsive pattern. In the times of the day when anxiety levels are low, the sustained vibration sent at a set programmed interval, such as every hour, provides the user with a short period of time in which he or she has an opportunity to observe his or her thoughts and feelings, identify the negative ones (before they are fully charged) and consciously and intentionally begin to change them. The treatment of addictions and compulsive behaviors requires constant observance and monitoring of the emotions, thoughts, feelings and behaviors that empower and trigger them. Together the device and method of the present invention provides the means necessary to accomplish this in a complete manner, offering monitoring and tracking of sensory feedback as well as biofeedback from internal processes.

In the case of athletes and fitness enthusiasts looking for ways to naturally increase the flow and quality of energy into their systems, the device provides both a tool and method that allows them to deliberately direct and manage the quality and intensity of energy input and output. The device and method accomplish this by getting the user acquainted with the dynamics of body/mind processes involved in the generation and conservation energy, increasing the user's ability to effectively identify the way in which energy is misdirected and wasted, including energy expended in the experiencing of unconscious negative emotions and the corresponding physical responses to negative emotions that affect specific parts of the body, causing those body parts to go weak affecting performance. Biofeedback therapy is often used by athletes to enhance performance and an athlete will benefit greatly from effectively identifying the parts of the body affected by the experience of a negative heightened emotion, through the use of the present device and method. Biofeedback in the present invention can be used as a reverse process, where the physiological response, resulting from stress, negative thoughts or emotions is treated from the outside in. By consciously and intentionally focusing the energy of the mind on relaxing the part of the body affected, the athlete is capable of controlling and reversing the stress or negative response that is affecting performance.

For individuals wanting to find balance, have more clarity of mind, and experience an expanded sense of awareness and well-being, the use of the device by means of its sustained physical stimulus creates the mental space and the condition necessary to the experiencing of awareness. The device's stimulus draws the user's mind's attention to his or her body, stopping the user's mind's usual activity for a short period of time creating a space to observe and re-evaluate the user's environment and his or her thoughts about it. Drawing the mind out its compulsive thought patterns naturally puts it into a more centered relaxed state beneficial for the overall wellbeing of its users.

### BRIEF DESCRIPTION OF THE DRAWING

While the specification concludes with claims particularly pointing out and distinctly claiming the present invention, it is believed the same will be better understood from the following description taken in conjunction with the accompanying drawings in which:

FIG. 1A is a perspective view illustrating a bottom view of an embodiment of the device of the present invention; and

FIG. 1B illustrates a side view of the embodiment of the device of the present invention of FIG. 1A; and

FIG. 1C illustrates a side view cross section of the embodiment of the device of FIG. 1A; and

FIG. 1D displays a perspective cross section of an embodiment of the device of the present invention; and

FIG. 1E illustrates a perspective assembly view of a possible embodiment from the top of the device of the present invention; and

FIG. 2A displays a perspective bottom view of a possible embodiment from the bottom of the device of the present invention; and

FIG. 2B displays a perspective top view of a possible embodiment from the top of the device of the present invention; and

FIG. 2C displays a perspective cross section view of a possible embodiment from the top of the device of the present invention; and

FIG. 3A displays a bottom perspective view of the preferred embodiment of the device of the present invention; and

FIG. 3B displays a top perspective view of the preferred embodiment of the device of the present invention; and

FIG. 3C displays magnified focus of the magnet 2 on the side of FIG. 3A; and

FIG. 3C1 displays magnified focus of the magnet 2 assembly on the side of FIG. 3A; and

FIG. 3D displays magnified focus on a control button of FIG. 3A; and

FIG. 3E displays the battery 3 and device casing 8 of FIG. 3B; and

FIG. 3E1 displays assembly of battery 3, the battery opening 6 and device casing 8 of FIG. 3B; and

FIG. 4A is a representation of the device of the present invention adapted to be worn on the wrist of a user; and

FIG. 4B displays the device attachment 14 of FIG. 4A; and

FIG. 5A is a representation of the device of the present invention adapted to be worn on the wrist of a user; and

FIG. 5B displays an embodiment of the wristband attachment of FIG. 5A displaying the band features 18; and

FIG. 6A is a representation of the device of the present invention adapted to be worn on the upper arm of a user; and

FIG. 6B displays the band features 18 of FIG. 6A; and

FIG. 6C displays the attachment portion of FIG. 6A securing the vibration plate 4 to the skin of the user to detect biofeedback; and

FIG. 7A displays an inside perspective top view of an embodiment of the frame 5 of the device of the present invention; and

FIG. 7B displays a view of the frame 5 of FIG. 7A as viewed from the side thereof; and

FIG. 7C displays a cross section of the frame 5 of device of FIG. 7A as viewed from the side thereof; and

FIG. 7D displays an outside bottom view of an embodiment of the frame 5 of the device of the present invention; and

FIG. 7E displays a cross section of the of the frame 5 of FIG. 7D as viewed from the side thereof; and

FIG. 7F is a top view of the frame 5 of the present invention as viewed from the inside thereof;

FIG. 7G is a cross section of the frame 5 of FIG. 7F sectioned between the two magnets 2 as viewed from the side thereof; and

FIG. 7H is a cross section of the frame 5 of FIG. 7F wherein the cross section is taken through a hole 7 to receive a control button 1, as viewed from the side thereof; and

FIG. 71 is a perspective view of the cross section of FIG. 7H, as viewed from the top at an angle of 45 degrees thereof; and

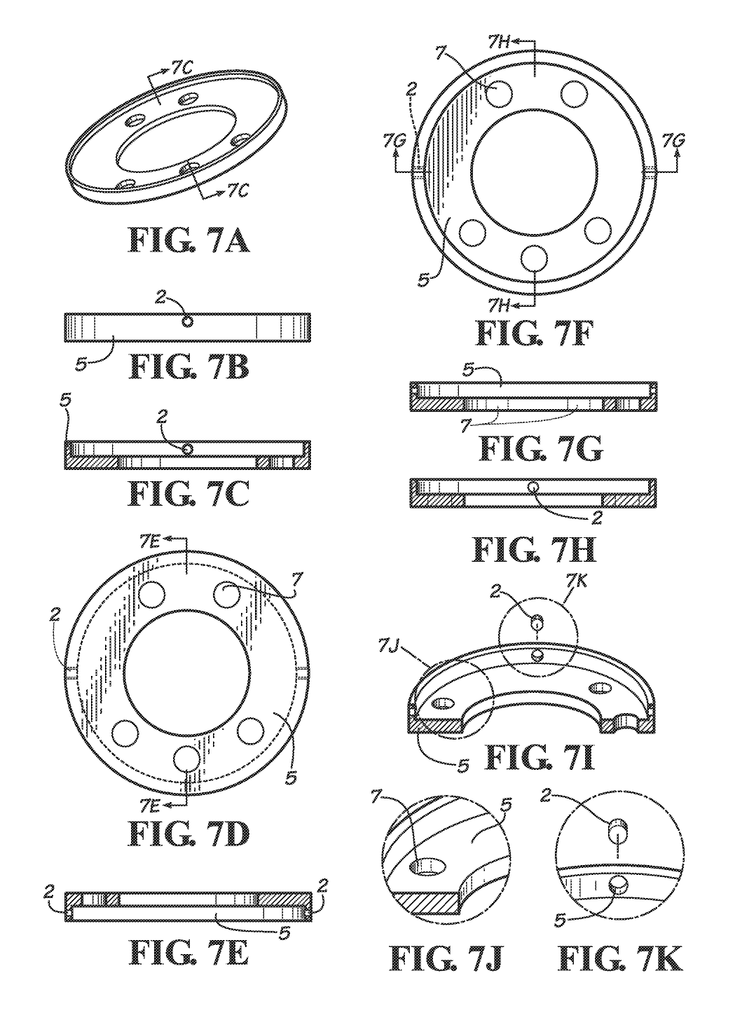

FIG. 7J is a perspective view of the inside of the device frame 5 of FIG. 7I displaying the hole 7 to receive a control button 1; and

FIG. 7K is a perspective view of the inside of the device frame 5 of FIG. 7I displaying the hole to receive a magnet 2; and

FIG. 8A displays a perspective view of the outside top front of the preferred wrist band attachment and device of the present invention; and

FIG. 8B displays a perspective view of the inside bottom view of the device in the preferred wrist band attachment of the present invention as viewed from the back of the band 18 attachment; and

FIG. 8C displays the flexible fabric band 18 connecting a contouring steel laminate 17 covered by a fabric 15 of FIG. 8A; and

FIG. 8D displays the contouring steel laminate 17 covered by a fabric 15 of FIG. 8A; and

FIG. 9A displays a perspective view of the preferred embodiment of the wrist band embodiment of the present invention as viewed from the top to display how the device is secured into the flexible ring 14 of the wristband 16; and

FIG. 9B illustrates a magnified cross section view of the wristband attachment 16 of FIG. 9A, to reveal a top rubber flap 20 connecting to the flexible rubber ring 14, and the band is made of an inner contouring steel laminate 17 covered by a fabric 15; and

FIG. 9C illustrates a magnified cross section view of the wristband attachment 16 of FIG. 9A, to view the inside wall 19 of the flexible rubber ring 14 which receives the device; and

FIG. 9D displays an external top view of the wristband 16 revealing external parts including a top rubber flap 20 connecting to the flexible rubber ring 14 and the outer fabric 15 covering; and

FIG. 9E displays an internal cross section side view of the wristband 16 revealing internal parts including a flexible fabric 18, and an inner layer of steel laminate 17 which contours the wrist; and

FIG. 9F illustrates the interval view of the wristband 16 attachment; and

FIG. 9G displays a close up of the wristband 16 attachment of FIG. 9F with a top rubber flap 20 connecting to the flexible rubber ring 14, and the band is made of an inner contouring steel laminate 17; and

FIG. 10 is a representation of the device of the present invention adapted to be worn from a connection 22 around the neck of a user to be secured to the chest of a user, and displays an embodiment of the attachment of the present invention; and

FIG. 11 is a representation of the device of the present invention adapted to be worn from a connection 22 around the neck of a user to be secured to the abdomen of a user, and displays an embodiment of the attachment of the present invention; and

FIG. 12 is a flow diagram representing an embodiment of the method of the present invention; and

FIG. 13 is a diagram of the interfaces, function and components of the preferred embodiment of the present wearable electronic device; and

FIG. 14 is a flow diagram representing an embodiment of device activation method of the present invention.

### DETAILED DESCRIPTION OF THE INVENTION

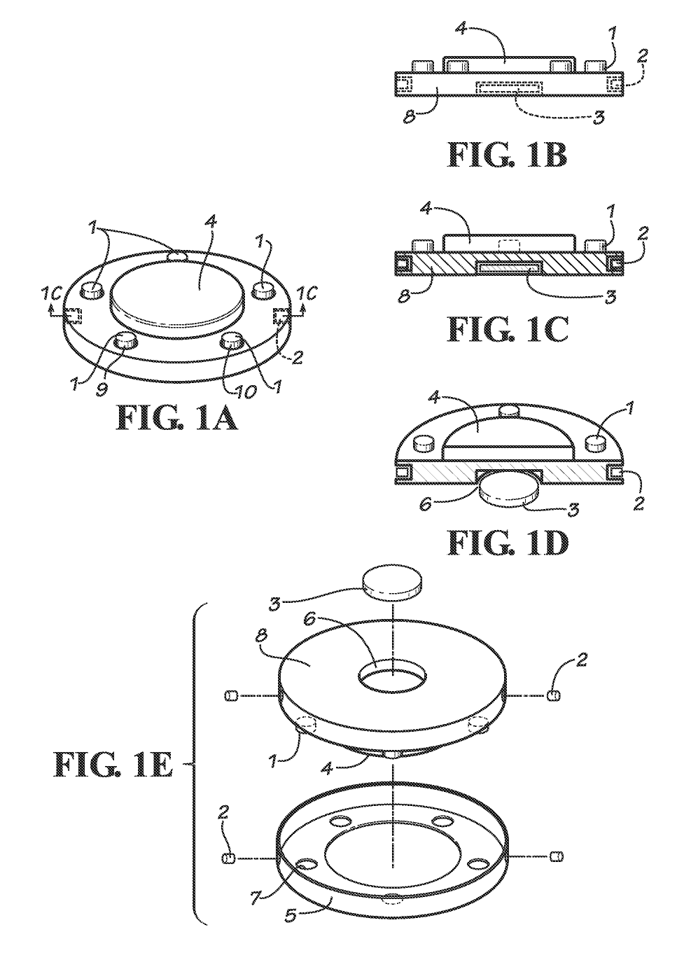

Referring now to the invention in more detail, FIGS. 1A-1E show multiple perspective views of a possible embodiment of the device of the present invention, containing a bottom view, top view, cross section view and an assembly view. In this embodiment, the bottom of the device as is displayed in FIG. 1A contains control buttons 1, magnets 2, and a vibration plate 4. The control buttons 1 allow the user to turn controls on and off or to adjust the controls of the device. In the preferred embodiment of the present device one control button 1 would turn off and on the thermal sensor 9, and another control button 1 would turn the preprogrammed interval notification off and on 10. Other control buttons 1 would control settings of the device, such as the duration of the vibration, intensity of vibration, and adjust the interval length of time between vibrations. Although the preferred embodiment of the device alerts the user through a vibration, one of ordinary skill in the art would understand that other physical stimulation means could be used in this device to the same effect.

The vibration plate 4 is disposed on the outside of the device so that it comes in to direct contact with the user's skin. The vibration plate 4 transmits a vibration to the user's skin when activated. Activation is based on a time interval being met or in response to a sensory cue. In the preferred embodiment, the device would come with a pre-programmed interval setting for one hour, wherein after every hour the device would send a vibration to the user via the vibration plate 4. In the preferred embodiment, the device will register and respond to external body temperatures in the range of 91.4 to 86 degrees Fahrenheit, as these external body temperatures are indicative of the presence of a stressed state for a healthy individual under stable conditions. A healthy individual under stable conditions is defined herein to refer to an individual of good health, not taking medications that alter the body temperature, in the standard environment of 77 degrees Fahrenheit, and that the individual is not engaged in physical activity that will effect body temperature. Temperatures below 91.4 will activate the vibration to be transferred to a user via the vibration plate 4. The magnets 2 of the device assist in securing the device to a wearable item, such that the vibration plate 4 will come into intimate contact with a user's skin.

FIG. 1B further displays a side view of the device, with control buttons 1, magnets 2, the battery 3 for operation of the device, the device casing 8 and the vibration plate 4. FIG. 1C is a side view cross section of the device where the cross section is taken through the magnets 2, and displays device control buttons 1, magnets 2, battery, the device casing 8 and the vibration plate 4. Further, FIG. 1D provides a perspective cross section view of the top of the device revealing the battery 3, the battery opening 6, device casing 8 and magnets 2 for securing the device to attachments to be worn by the user. The device casing 8 includes the electric circuit board of the device. Finally, Figure lE displays the assembly of the device of the present invention. As illustrated in FIG. 1E, the device frame 5, with holes 7 for the control buttons 1 to pass through, connects with the vibration plate 4 and control buttons 1, while the magnets 2 attach on the sides of the device and the battery 3 fits into the battery opening 6 and is covered by the casing top covering 8.

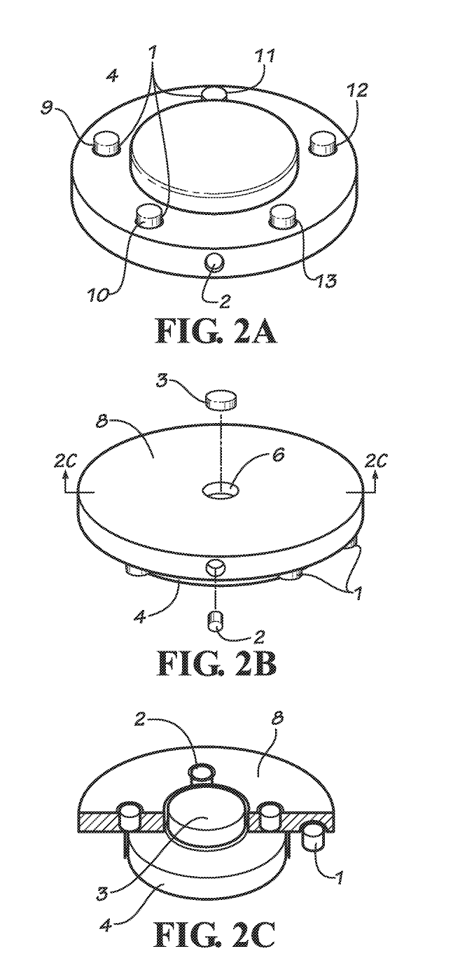

FIGS. 2A-2C provides additional views of the device. As is displayed in FIG. 2A, the vibration plate 4 and control buttons 1 are on the bottom of the device, while the magnets are on the sides of the device. In the preferred embodiment, the control buttons 1 contain an on/off control button for the thermal sensor component 9, and an on/off control button for the pre-programmed interval timer component 10. Further, in the preferred embodiment, the other control buttons, 11, 12 and 13 correspond to the control of the duration of the vibration, intensity of vibration, and adjust the interval length of time between vibrations. The top of the device, as illustrated in FIG. 2B includes the battery 3, fitting into the battery opening 6 and is contained within the casing 8 holding the circuit board. FIG. 2C provides a cross section of the device displaying the casing 8, battery 3, vibration plate 4, control buttons 1 and magnet 2.

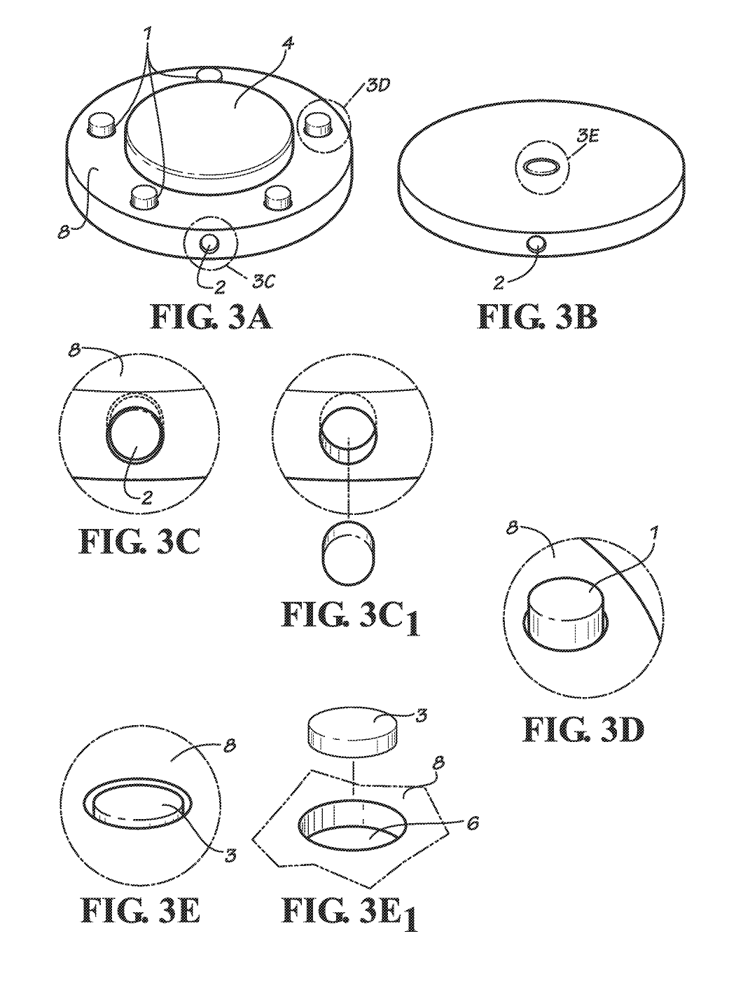

FIG. 3A displays a bottom perspective view of the device with control buttons 1, vibration plate 4 and magnets 2 disposed in the casing 8. FIG. 3B displays a top perspective view of the device with the battery 3 which inserts into the battery hole 6, magnets 2 for attachment to a wearable item and device casing 8. FIGS. 3C and 3C1 provide close up views of a magnet 2 in the casing 8, and how the magnet 2 would fit into the device. The drawing of FIG. 3D provides a close up of a control button 1 fitting into the casing 8. As the top of the device is also displayed in FIG. 3B, with the battery 3, magnet 2 and casing 8. A close up of the battery 3 fitting into the battery opening 6 of the device casing 8 is shown in FIGS. 3E and 3E1.

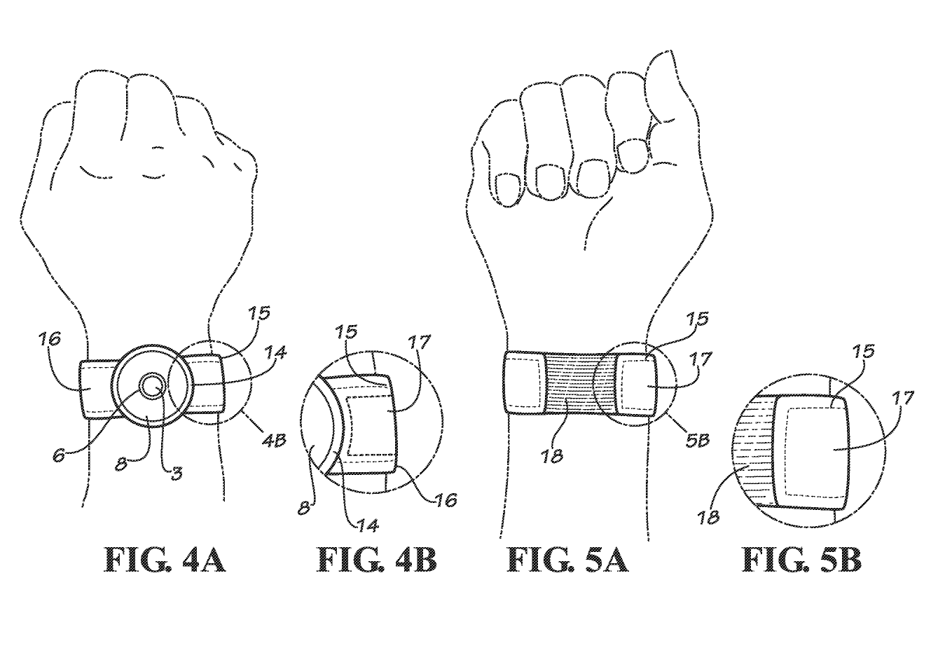

FIG. 4A provides a view of the device from the top secured to a wristband attachment 16. The device, with the battery 3, in the battery opening 6, secured within the casing 8, is attached to a flexible rubber ring 14 of the wristband 16 which holds the device in place on the wristband 16. The flexible rubber ring 14 can be bent to place the device inside, and the magnets 2 on the device firmly hold the device in alignment with receiving magnets inside the rubber ring 14. In the preferred embodiment, as displayed in the close up view of FIG. 4B, the rubber ring 14 is bound to the wristband 16, which has an inner layer of steel laminate 17 which contours the wrist, and is covered with a fabric 15. The fabric displayed FIG. 4B has a decorative stitching. One of ordinary skill would know this is only one of many ways the device can appear, and is enlarged for displaying details. FIG. 5A displays an alternate view of the user wearing the wristband attachment 16. The wristband which has an inner layer of steel laminate 17 which contours the wrist, and is covered with a fabric 15 is connected with a flexible fabric band 18. The flexible fabric band 18 may be made of an elastic fiber or another stretchable material to stretch to fit a multitude of users with varying wrist sizes. FIG. 5B provides a close up view of the inner layer of steel laminate 17 of the wristband device which contours the wrist, is covered with a fabric 15 and connects to the flexible fabric band 18 of the wristband attachment.

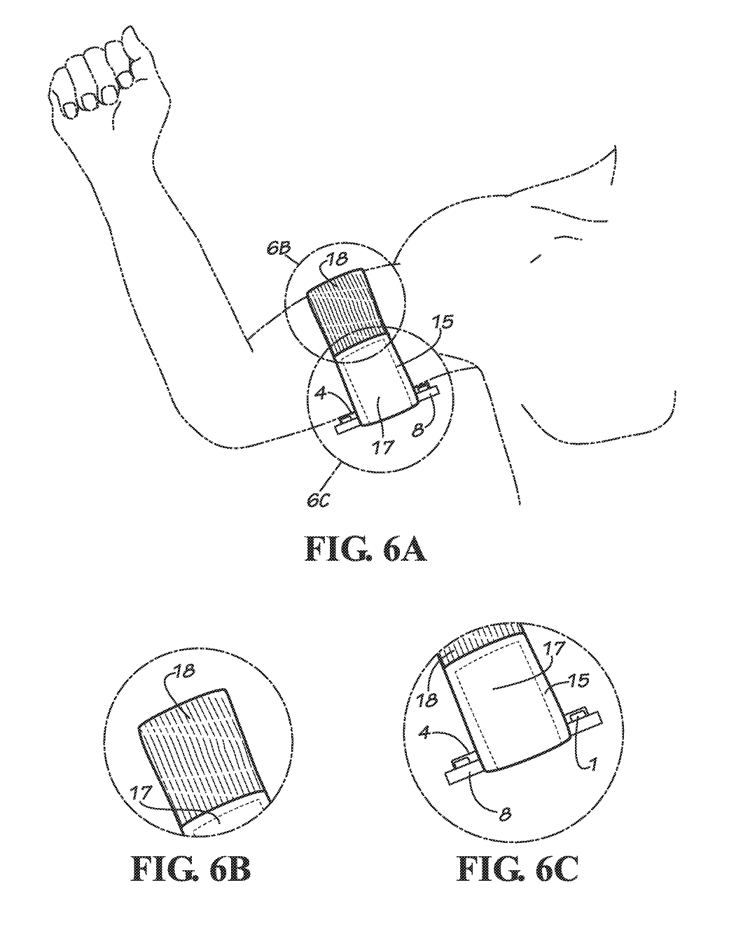

The wristband attachment 16 displayed in FIGS. 4 and 5 can also be worn on the upper arm of a user, as is displayed in FIG. 6A. This is due to the flexible fabric band 18. FIG. 6A displays the inner layer of steel laminate 17 which contours the wrist, and is covered with a fabric 15, connected with a flexible fabric band 18, while the device is attached so that the bottom containing control buttons 1, and vibration plate 4, is put into contact with the user's upper arm and the top of the device with the casing top covering 8 is exposed to the air. FIG. 6B displays a close up of the elastic part of the band features 18 that provides the necessary stretch to be worn either in the wrist or the upper arm. FIG. 6C displays a close up of the attachment portion where the device is attached to the armband, securing the vibration plate 4 to the skin of the user to detect biofeedback. In FIG. 6C, the device is attached so that the bottom of the device including control buttons 1, and vibration plate 4, is placed into direct contact with the user's upper arm, with casing 8 outward, and the armband itself contains an inner steel layer 17 covered in fabric 15 connected to a flexible fabric band 18 to comfortable fit a wide range of upper arm sizes.

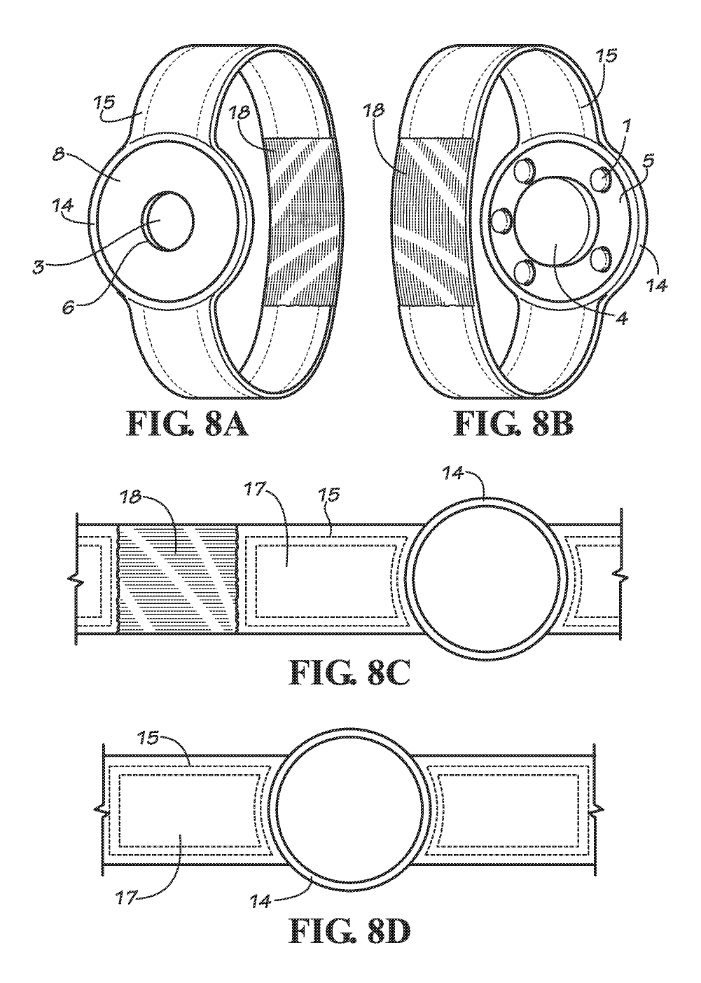

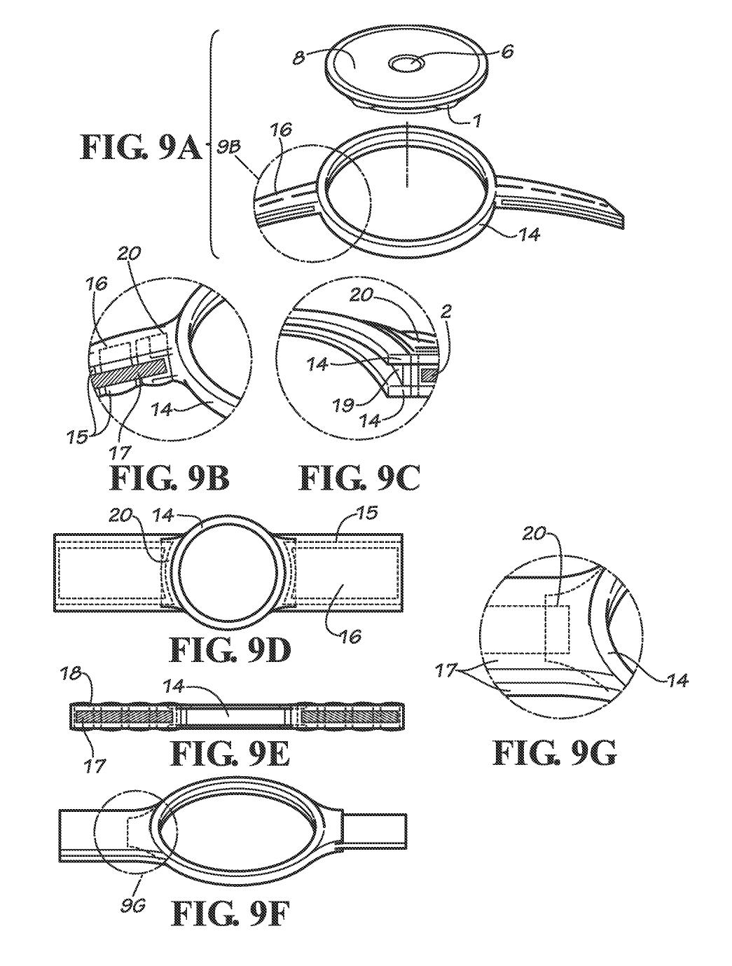

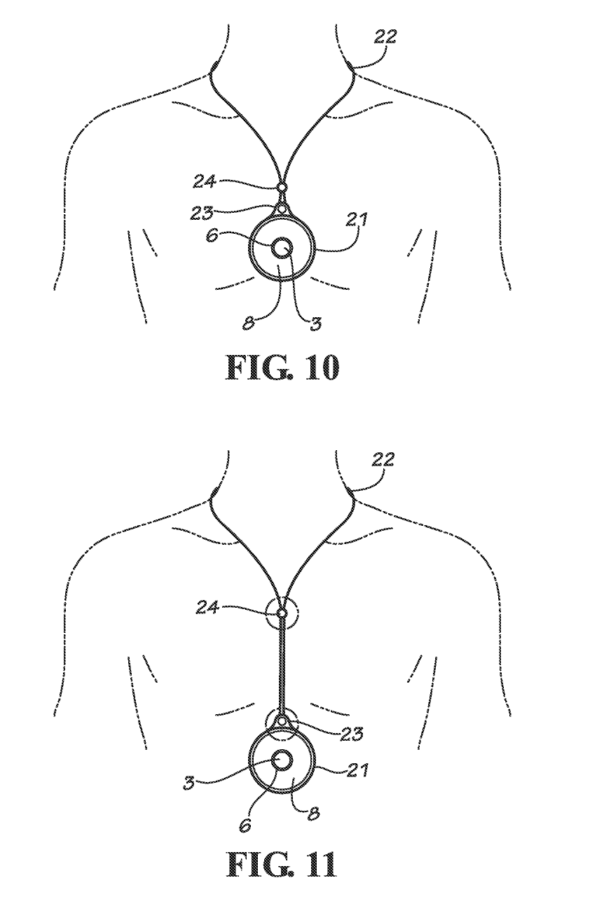

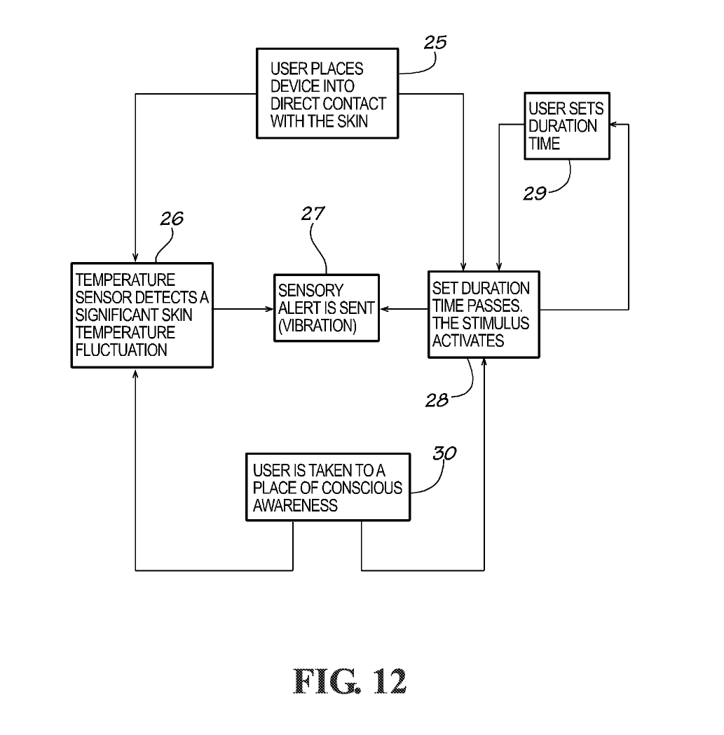

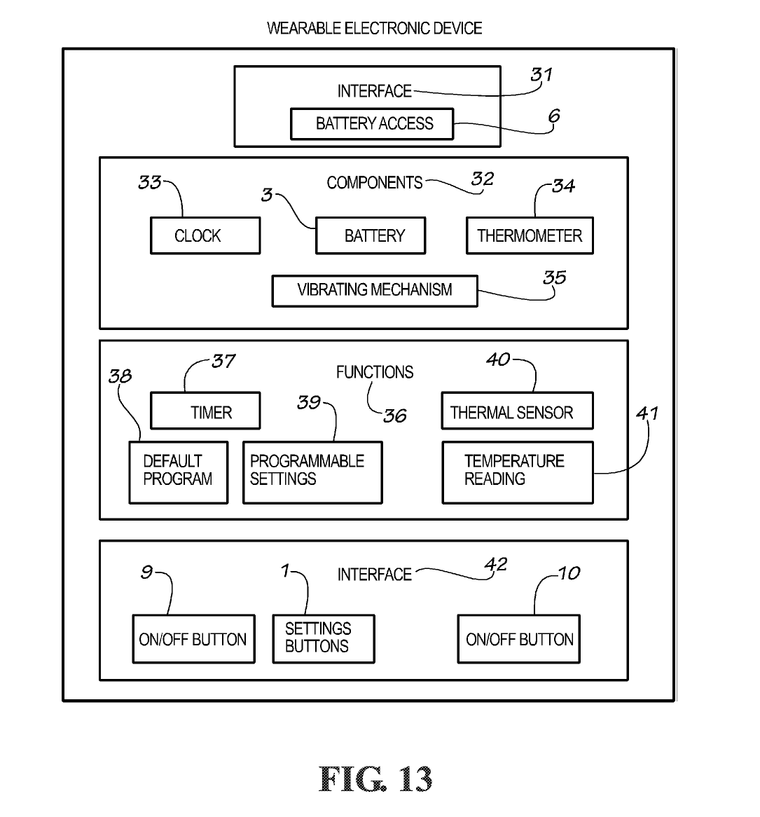

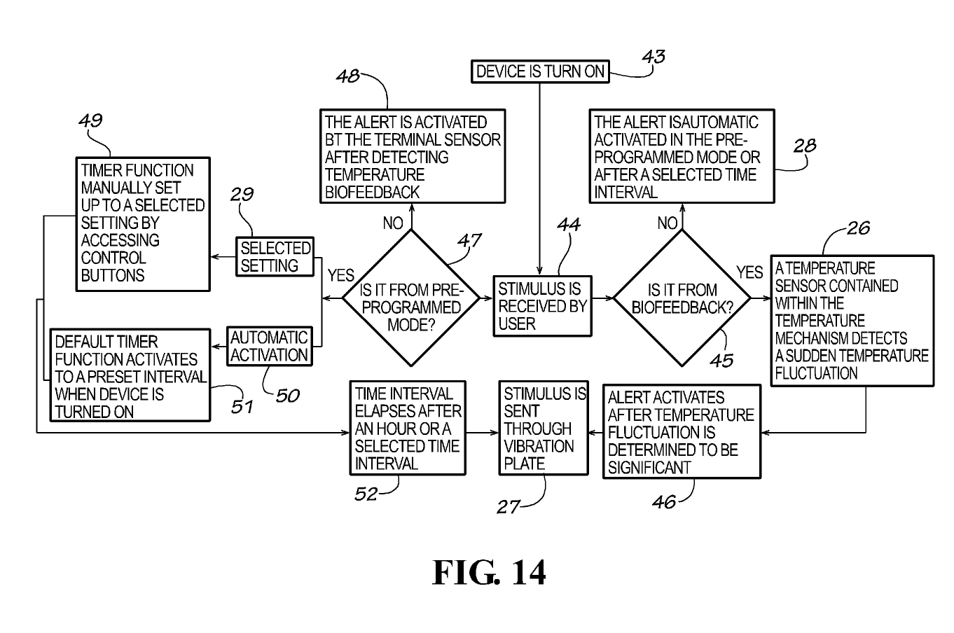

FIGS. 7A-7K display the device frame 5 with holes 7 for the control buttons to fit through and magnets 2 to assist in attachment to wearable pieces, such as the wristband. FIG. 7A displays an inside perspective top view of the device frame 5. FIG. 7B displays the device frame 5 from the side view, with magnets 2, and FIG. 7C is a side cross section view of the frame 5 with magnets 2. FIG. 7D displays an outside bottom view of the device frame 5 with holes 7 for the control buttons to fit through and magnets 2, and FIG. 7E is a side cross section of FIG. 7D. FIG. 7F provides an inside top view of the device frame 5 revealing control button holes 7 and magnets 2. FIG. 7G is a cross section of the frame 5 of FIG. 7F sectioned between the two magnets 2 as viewed from the side thereof, whereas FIG. 7H is a cross section of the frame 5 of FIG. 7F wherein the cross section is taken through a hole 7 to receive a control button 1, as viewed from the side thereof to view magnets 2. FIG. 7H provides a cross section of the interior of the frame 5. A close up of the hole 7 in the frame 5 to receive control buttons is displayed in FIG. 71, whereas a close up of the magnet 2 fitting into the device frame 5 is provided in FIG. 7K.

FIGS. 8A-8D provide views of the wristband attachment 16 and device from different angles. The wristband 16, includes a flexible fabric 18, and an inner layer of steel laminate 17 which contours the wrist covered with a fabric 15 and holding the flexible rubber ring 14 which the device slips into securely. FIG. 8 shows the device inside the rubber ring 14 from the inside (FIG. 8B) and outside (FIG. 8A) of the wristband 16. As is viewable in FIG. 8A, the wristband has a flexible fabric 18 connecting a contouring steel laminate 17 covered by a fabric 15, which attaches the flexible rubber ring 14 that holds the top of the device outward with the battery 3 disposed in the battery opening 6 and covered by the device casing top covering 8, and holds the bottom of the device against the skin of the user. The bottom of the device contains the vibration plate 4, control buttons 1 and frame 5, so that the vibration plate comes directly into contact with the user's skin, as is viewable from FIG. 8B. FIGS. 8C and 8D display the flexible rubber ring 14, which the device is inserted and attached inside connected to a contouring steel laminate 17 covered by a fabric 15 flexible fabric. FIG. 8C further displays the flexible fabric band 18 used to secure around the user's wrist, upper arm or ankle.

FIGS. 9A-G display multiple perspective views of the preferred embodiment of the wrist band embodiment of the present invention from the top, side and sectional views. FIG. 9A provides a perspective cross section view of the assembly of the wrist band attachment as viewed from the top to display how the device, containing control buttons 1 and battery opening 6 in the casing 8, is secured into the flexible ring 14 of the wristband 16 and held securely in place. The device, shown from the top displaying the battery 3, battery opening 6 and casing top covering 8, where the control buttons 1 and the vibration plate 4 are facing downward, fits into the flexible rubber ring 14 of the wristband 16. A close up of the wristband 16 in FIG. 9B reveals a top rubber flap 20 connecting to the flexible rubber ring 14, and the band is made of an inner contouring steel laminate 17 covered by a fabric 15, which can be decorated, such as with a stitching. A view of the flexible rubber inside wall 19 viewable in FIG. 9C of the rubber ring 14 also displays the magnetic connector 2 in the wristband which secures the magnets of the device through magnetism. FIG. 9D displays an external top view of the wristband 16 revealing external parts including a top rubber flap 20 connecting to the flexible rubber ring 14 and the outer fabric 15 covering. FIG. 9E displays an internal cross section side view of the wristband 16 revealing internal parts including a flexible fabric 18, the flexible rubber ring 14 to attach the device, and an inner layer of steel laminate 17 which contours the wrist. FIG. 9F illustrates the interval view of the wristband 16 attachment and FIG. 9G displays a close up of the wristband 16 attachment of FIG. 9F with a top rubber flap 20 connecting to the flexible rubber ring 14, and the band is made of an inner contouring steel laminate 17.

FIG. 10 is a representation of the device of the present invention adapted to be worn from a connection around the neck of a user to be secured to the chest of a user, and displays an embodiment of the attachment of the present invention. The drawing shows the device, with the vibration plate against the wearer's skin, and with the battery 3, in the battery opening 6, secured in with the covering 8 facing out from the wearer's body. The device is secured inside a flexible ring 21 so the device can be worn on the chest. The ring 21, is flexible and bendable for insertion of the device and contains complementary magnets to the device to form a secure hold. The device and ring 21 are suspended 23 from a chain, string, cord 22 or the like, where the length of the chain can be adjusted by extender(s) 24, which locks the device in the desired location for the user. FIG. 11 is a representation of the device of the present invention adapted to be worn from a connection around the neck of a user to be secured to the abdomen of a user, and contains all of the same elements as FIG. 10, however, the location of the device has been adjusted by the extender 24 to be worn on the user's abdomen.

The method of the present invention is described in the flow diagram of FIG. 12. First, the user places the device into direct contact with the user's skin 25. Then either one of two different paths can activate a sensory signal that will be sent to the user via the device. The user can use each feature separately, or in combination. When a temperature sensor detects a significant temperature fluctuation in the user's skin 26, a sensory alert is sent to the user 27. In the preferred embodiment of the present invention, the sensory alert will be in the form of a vibration. The sensory alert takes the user to a place of conscious awareness 30. Another path can activate the sensory alert 27, through a preprogrammed component, wherein a set duration of time passes and the stimulus activates 28, sending the sensory alert to the user 27 taking the user to a place of conscious awareness 30. Although the device is set to a default time interval, the user may adjust the interval duration 29.

FIG. 13 is a diagram of the interfaces, function 36 and components 32 of the present wearable electronic device. The interface of the top of the device 31 has a hole for battery access 6. The device components 32 include a timing element, such as a clock 33, an energy source, preferably a battery 3, a temperature reading component or thermometer 34 which can be in the form of a thermistor, and a stimulus generating mechanism, which in the preferred embodiment of the present invention is a vibrating mechanism 35. Herein disclosed, the timing element of the present device and method can be a real time clock, a chronometer, a stopwatch like timer, or any other timing mechanism that can be used to accurately track intervals of time.

The present device has multiple functions 36, as displayed in FIG. 13. Of these functions are the timer 37, a default program 38, as well as programmable settings 39. Further the device has a thermal sensor 40 and temperature reading 41 functions.

The interface of the bottom of the device 42 contains setting buttons 1, also referred to herein as control buttons. In the preferred embodiment as displayed in FIG. 13, the control buttons 1 contain an on/off control button for the thermal sensor component 9, and an on/off control button for the pre-programmed interval timer component 10. Further, in the preferred embodiment, the other control buttons, correspond to the control of the duration of the vibration, intensity of vibration, and adjust the interval length of time between vibrations.

FIG. 14 displays the preferred method of use of the present invention. When the device is turned on 43, a stimulus will ultimately be received by the user 44. In reviewing the present method, the first question evaluates whether the stimulus generated was in response to biofeedback 45. If not, the alert was automatically generated in the pre-programed mode or after an interval of time selected by the user 28. If the stimulus was generated in response to biofeedback, stimulus generation is due to the following process: a temperature sensor contained within the temperature mechanism detects a sudden temperature fluctuation 26, and the alert activates after the temperature fluctuation is determined to be significant 46, such that in the preferred embodiment, the stimulus is sent to the user 27 through the device vibration plate, wherein the user will receive the stimulus 44.

When the device is turned on 43, a stimulus will ultimately be received by the user 44, wherein the second question is: did the stimulus originate from the pre-programmed mode 47 of the device? If the answer is that the stimulus did not originate from the pre-programmed feature of the device, then the alert was activated by the thermal sensor after detection of temperature biofeedback 48. If however, the stimulus did originate from the pre-programmed mode, the user could have selected a setting 29, where the timer function was manually set by the user through use of control buttons to the preferred duration interval 49 or the device could have automatic activation 50 where the device activates to a preset interval when in operation 51. In either of these two pre-programmed activation methods, a time interval will elapse 52 at which point the stimulus is activated and sent to the user through the vibration plate 27, such that the user will receive the stimulus 44.

The advantages of the present invention include, without limitation providing a device and method for use in focusing attention and energy, to alert the user of a potential emotional disturbance and to provide the user with a reminder to clear his or her mind and allow for a new sense of awareness in the present moment. By combining stimuli sent at a set time interval with stimuli sent in response to a physiological change in the body, detected by change in body temperature, the device provides unique opportunities for users to be reminded to refocus energy in the present moment, which can have a variety of advantages from stress reduction to enhancing athletic performance.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. 1. A portable electrical device functioning locally for focusing energy and attention comprising an electric circuit (8), an energy source (3), a timing element, a discreet physical stimulus generator (4) and a programmable component, said locally functioning device characterized by
(a) at least one thermal sensor (9) wherein said thermal sensor(s) (9) is disposed to come into direct contact with the skin of the wearer user and includes a measurement component that measures and detects sudden changes in skin temperature, and
(b) wherein said physical stimulus generator (4) is coupled with said measurement component of said thermal sensor (9), such that a physical stimulus is sent to the wearer user if said measurement component detects a sudden change in skin temperature outside the range of normal, and
(c) said programmable component is coupled with said timing element and said physical stimulus generator (4), wherein said programmable component contains a default mode at which a physical stimulus is sent to said wearer user in pre-programmed intervals, and a programmable component, at which a physical stimulus is sent to the user at an interval programmed by said user.

2. The device of claim 1 wherein said thermal sensor(s) (9) is a thermistor.

3. The device of claim 1 wherein said physical stimulus generator is in the form of a vibration plate (4) disposed on the outside of said device so that said vibration plate (4) comes into direct contact with the user's skin, so when activated it provides a vibration notification to said user without the use of screens or visual stimulation.

4. The device of claim 1 wherein said measurement component of said thermal sensor (9) registers and responds to external body temperatures in the range of 91.4 to 86 degrees Fahrenheit, as these external body temperatures are indicative of the presence of a stressed state for a normal healthy individual under stable conditions.

5. The device of claim 1 further comprising control buttons (1), magnets (2) to assist in securing said device to a wearable item, and a vibration plate (4) as said physical stimulus generator, wherein control buttons (1) allow the user to turn controls on and off or to adjust the controls of the device, such that one control button (1) would turn off and on the thermal sensor (9), and another control button (1) would turn the preprogrammed interval notification off and on (10), and other control buttons (1) would control settings of the device, such as the duration of the vibration (11), intensity of vibration (12), and adjust the interval length of time between vibrations (13).

6. The device of claim 1 further comprising a flexible attachment band (18) with a flexible rubber ring (14) to receive and hold said device in place with magnets (2) such that the thermal sensor (9) and physical stimulus generator (4) of said device are in direct contact with the user's skin and wherein said flexible rubber ring (14) has an inner layer of steel laminate (17) which contours around a body part of the user and is covered with a fabric (15), wherein said attachment band can be in the form of a wristband (16), ankle band or band worn on the upper arm.

7. The device of claim 1 further comprising a flexible ring (21) to receive and hold said device in place with magnets (2) and a chain, string or cord (22) attached to said flexible ring (21) where the length of the chain can be adjusted by extender(s) (24) which locks said device and flexible ring (21) in the desired location of the user's chest or abdomen, such that the thermal sensor (9) and physical stimulus generator (4) of said device are in direct contact with the user's skin and wherein said flexible ring (21) has an inner layer of steel laminate (17) which contours around a body part of the user and is covered with a fabric (15).

8. A method of focusing energy and attention of a user comprising the steps of: said user wearing a device that functions locally with at least one thermal sensor (9), an electric circuit (8), an energy source (3), a timing element, a discreet physical stimulus generator (4) and a programmable component in direct contact with the user's skin (25), measuring the skin temperature of said user through said device, detecting sudden significant temperature fluctuations outside the normal range (26) and transmitting to said user a physical stimulation (27) in response to temperature fluctuations outside the range of normal, wherein said functions of measuring, detecting and transmitting are all occurring locally within the device.

9. The method of claim 8, further comprising the steps of activating the programmable component of said device, such that a physical stimulus is transmitted to said user (27) over defined intervals of time (28), wherein duration of said intervals may be adjusted by said user (29).

10. The method of claim 8 wherein said measurement component of said thermal sensor (9) sends a physical stimulus (27) to said user when said thermal sensor (9) detects external body temperature fluctuations bringing the body temperature below 91.4 degrees Fahrenheit, which may be indicative of a potential heightened negative emotion.

11. The method of claim 8 wherein said user wears said device on the wrist, ankle or upper arm, by fastening said device inside a flexible attachment band (18) with a flexible rubber ring (14) to receive and hold said device in place with magnets (2) such that the thermal sensor (9) and physical stimulus generator (4) of said device are in direct contact with the user's skin and wherein said flexible rubber ring (14) has an inner layer of steel laminate (17) which contours around a body part of the user and is covered with a fabric (15), wherein said attachment band can be in the form of a wristband (16), ankle band or band worn on the upper arm.

12. The method of claim 8 wherein said user wears the device on the chest and abdomen by fastening said device inside a flexible ring (21) designed to receive and hold said device in place with magnets (2) and adjusting a chain, string or cord (22) attached to said flexible ring (21) where the length of said chain can be adjusted by extender(s) (24) which locks said device and flexible ring (21) in the desired location of the user's chest or abdomen, such that the thermal sensor (9) and physical stimulus generator (4) of said device are in direct contact with the user's skin.

13. A method of assisting a user in stress management, developing problem solving skills, overall performance enhancement, assisting in addiction control, controlling compulsive behaviors, dealing with anxiety, increased athletic performance, centering balance, clarity of mind, and/or seeking to experience an expanded sense of awareness and well-being by focusing energy and attention comprising said user placing a locally functioning device with at least one thermal sensor (9), an electric circuit (8), an energy source (3), a timing element, a discreet physical stimulus generator (4) and a programmable component in direct contact with the user's skin (25), measuring the skin temperature of said user through said device, detecting significant sudden temperature fluctuations outside the normal range (26) internally in the device and transmitting to said user a physical stimulation (27) via said device in response to significant temperature fluctuations outside the range of normal, placing the user in a state of conscious awareness (30) and said user using said device in default mode or selecting the time interval (29) at which a physical stimulation (27) is sent to said user, such that when said interval of time has passes the physical stimulus generator (4) activates (28) transmitting a physical stimulation (27) to said user, placing the user in a state of conscious awareness (30).

14. The method of claim 13 wherein said measurement component of said thermal sensor (9) sends a physical stimulus (27) to said user when said thermal sensor (9) detects external body temperature fluctuations bringing the external body temperature below 91.4 degrees Fahrenheit, which may be indicative of a potential heightened negative emotion.

15. The method of claim 13 wherein said user wears said device on the wrist, ankle or upper arm, by fastening said device inside a flexible attachment band (18) with a flexible rubber ring (14) to receive and hold said device in place with magnets (2) such that the thermal sensor (9) and physical stimulus generator (4) of said device are in direct contact with the user's skin and wherein said flexible rubber ring (14) has an inner layer of steel laminate (17) which contours around a body part of the user and is covered with a fabric (15), wherein said attachment band can be in the form of a wristband (16), ankle band or band worn on the upper arm.

16. The method of claim 13 wherein said user wears the device on the chest and abdomen by fastening said device inside a flexible ring (21) designed to receive and hold said device in place with magnets (2) and adjusting a chain, string or cord (22) attached to said flexible ring (21) where the length of said chain can be adjusted by extender(s) (24) which locks said device and flexible ring (21) in the desired location of the user's chest or abdomen, such that the thermal sensor (9) and physical stimulus generator (4) of said device are in direct contact with the user's skin.

17-20. (canceled)
